(19) United States
(12) Patent Application Publication
(10) Pub. No.: US 2013/0085534 A1
(43) Pub. Date: Apr. 4, 2013

(54) CONNECTORS FOR A SECONDARY BONE ANCHOR

(71) Applicants: Nicolas Hainard, Fontainemelon (CH); Frank Spratt, Lelocle (CH)

(72) Inventors: Nicolas Hainard, Fontainemelon (CH); Frank Spratt, Lelocle (CH)

(21) Appl. No.: 13/625,989

(22) Filed: Sep. 25, 2012

Related U.S. Application Data
(60) Provisional application No. 61/541,213, filed on Sep. 30, 2011.

(51) Int. Cl. A61B 17/70

(52) U.S. Cl. 606/278

(57) ABSTRACT

A connector for connecting a second bone anchor to a first bone anchor includes a first component engageable to the first bone anchor and rotatably adjustable relative to the first bone anchor, a second component connected to the first component and forming a socket to receive the second bone anchor in a plurality of angular orientations, and a closure mechanism engageable with the second component to fix the second bone anchor at a selected angular orientation relative to the second component.

### CROSS REFERENCE TO RELATED APPLICATIONS

This application claims priority to U.S. Provisional Patent Application No. 61/541,213, filed Sep. 30, 2011, incorporate herein by reference.

### BACKGROUND

Bone anchors may be used in orthopedic surgery to fix bone during the healing or fusion process. In spinal surgery, bone anchors may be used with spinal fixation elements, such as spinal rods, to stabilize multiple vertebrae either rigidly, in which no relative motion between the vertebrae is desired, and dynamically, in which limited, controlled motion between the vertebrae is desired. One problem with the use of bone anchors is that bone anchors may pullout or otherwise be displaced from the bone prior to the healing or fusion process completing. This problem is particularly common when a bone anchor is positioned in poor quality bone such as osteoporotic bone. This problem is also common when a bone anchor is placed in the sacrum at the end of a spinal construct. The generally low strength of the sacrum and the moment arm exerted on the sacral anchor results in increased risk of pulling out the sacral anchor. Accordingly, there is need for improved bone anchors that minimize instances of such anchor pull out.

### SUMMARY

Disclosed herein are connectors for connecting a secondary bone anchor, such as a bone screw, with a primary bone anchor, such as a polyaxial screw for coupling a spinal rod to a vertebra. The use of a secondary bone anchor connected by the connector to the primary bone anchor can minimize instances of the primary bone anchor pulling out of bone.

In accordance with one exemplary embodiment, a connector for connecting a second bone anchor to a first bone anchor includes a first component engageable to the first bone anchor and rotatably adjustable relative to the first bone anchor, a second component connected to the first component and forming a socket to receive the second bone anchor in a plurality of angular orientations, and a closure mechanism engageable with the second component to fix the second bone anchor at a selected angular orientation relative to the second component.

### BRIEF DESCRIPTION OF THE FIGURES

These and other features and advantages of the devices and methods disclosed herein will be more fully understood by reference to the following detailed description in conjunction with the attached drawings in which like reference numerals refer to like elements through the different views. The drawings illustrate principles of the devices and methods disclosed herein and, although not to scale, show relative dimensions.

FIGS. 1 and 2 are perspective views of a spinal system including a plurality of spinal rods, a plurality of primary bone anchors, a plurality of secondary bone anchors, and a plurality of exemplary connectors for connecting a secondary bone anchor to a primary bone anchor, illustrating the system connected to the spinal column including the sacrum;

FIG. 3 is a perspective view of one of the exemplary connectors of the spinal system of FIGS. 1 and 2, illustrating the connector connecting a secondary bone anchor to a primary bone anchor coupled to a spinal rod;

FIG. 4 is an exploded view of the components of FIG. 3, illustrating the connector, the secondary bone anchor, the primary bone anchor, and the spinal rod;

FIG. 5 is a side view in cross section of the components of FIG. 3, illustrating the connector, the primary bone anchor, and a spinal rod;

FIGS. 6 and 7 are a perspective views of the exemplary connector of FIG. 3;

FIG. 8 is a side view of the exemplary connector of FIG. 3;

FIG. 9 is a top view of the exemplary connector of FIG. 3;

FIG. 10 is side view in cross section of the exemplary connector of FIG. 3;

FIG. 11 is a side view of an exemplary secondary bone anchor;

FIG. 12 is a perspective view of an exemplary closure mechanism for the connector of FIG. 3;

FIG. 13 is a side view in cross section of the closure mechanism of FIG. 12;

FIGS. 14 and 15 are perspective views of another exemplary connector of the spinal system of FIGS. 1 and 2, illustrating the connector connecting a secondary bone to a primary bone anchor coupled to a spinal rod;

FIG. 16 is an exploded view of the components of FIGS. 14 and 15, illustrating the connector, the primary bone anchor, the secondary bone anchor, and the spinal rod;

FIG. 17 is a top view of the connector of FIGS. 14 and 15; and

FIG. 18 is a side view in cross section of the connector of FIG. 17.

### DETAIL DESCRIPTION OF EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

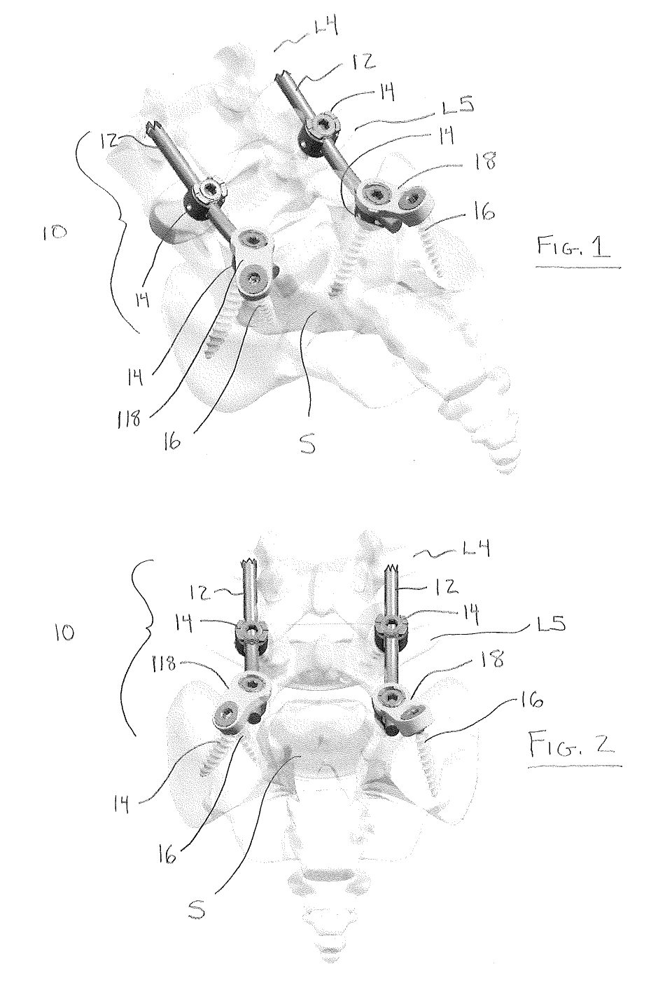

FIGS. 1-2 illustrate an exemplary embodiment of a spinal fixation system 10 including a plurality of spinal rods 12, a plurality of primary bone anchor assemblies 14 for connecting one of the spinal rods 12 to bone, a plurality of secondary bone anchors 16, and a plurality of connectors 18 and 118 for connecting a secondary bone anchor 16 to one of the primary bone anchor assemblies 14. The secondary bone anchor 16 connected to a primary bone anchor assembly 14 by a connector 18, 118 minimizes the pulling out of the primary bone anchor 14 from bone. As discussed in more detail below, the connectors 18, 118 permits inter-operative connection of a secondary bone anchor 16 to the primary bone anchor assembly 14. The connectors 18, 118 also permit the surgeon to adjust the secondary bone anchor 16 to a wide range of angular orientations relative to the primary bone anchor assembly 14 thereby by allowing the surgeon more flexibility to select the desired trajectory within the bone for the implantation of the secondary bone anchor 16.

The exemplary spinal fixation system 10 is particularly suited for sacral fixation to a patient's spine. As illustrated in FIGS. 1 and 2, in one exemplary spinal construct assembled from the exemplary spinal fixation system 10, primary bone anchor assemblies 14 may be implanted in one or more vertebra of the spine and the sacrum of the spine. For example, a primary bone anchor assembly 14, such as a polyaxial pedicle screw, may be positioned within the pedicles of the L5 vertebra and the sacrum S of the spine. Further primary bone anchor assemblies 14 may be positioned in the pedicles of other vertebra of the spine, e.g., L4 or L3, depending on the number of levels to be treated by the construct. A spinal rod 12 or other spinal fixation device may be used to connect the vertebra to be treated together and to the sacrum. The exemplary construct is a bilateral construct. Alternatively, a unilateral construct may be implanted using the spinal fixation system 10. Connectors 18, 118 can be used to supplement the fixation of the primary bone anchor assemblies 14 implanted in the sacrum S. Connectors 18, 118 permit the coupling of a secondary bone anchor 16 implanted in the sacrum to the primary bone anchor assembly 14 implanted in the sacrum and thereby provide additional pull out strength to the construct in the sacrum. Preferably, the second bone anchor 16 is oriented at a trajectory divergent to the trajectory of the primary bone anchor assembly 14 to maximize the pullout strength of the construct.

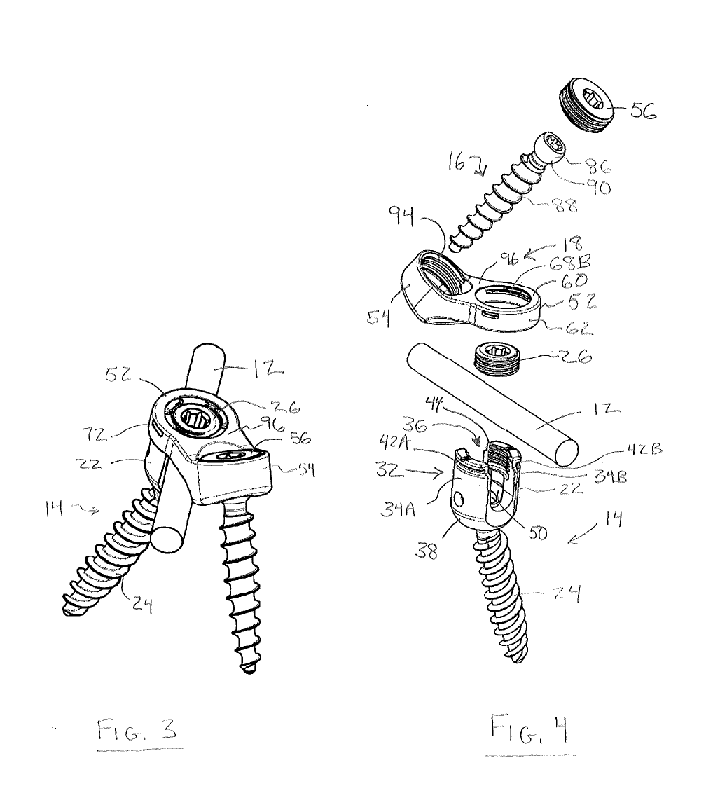

Referring to FIGS. 3-5, a primary bone anchor assembly 14 may include a proximal receiver member 22 for receiving a spinal fixation element, such as spinal rod 12, to be coupled to the bone anchor 14, a distal bone anchor 24 for engaging bone, and a closure mechanism 26 to capture a spinal fixation element within the receiver member 14 and fix the spinal fixation element with respect to the receiver member 22. The bone anchor 24 includes a proximal head 28 and a distal shaft 30 configured to engage bone. The receiver member 22 has a proximal end 32 having a pair of spaced apart arms 34A, 34B defining a recess 36 therebetween and a distal end 38 having a distal end surface 40 defining opening through which at least a portion of the bone anchor 24 extends. The closure mechanism 26 may be positionable between and may engage the arms 34A, 34B to capture a spinal fixation element within the receiver member 22 and fix the spinal fixation element with respect to the receiver member 22.

Continuing to refer to FIGS. 3-5, the proximal head 28 of the bone anchor 24 in the exemplary embodiment is generally in the shape of a truncated sphere having a planar proximal surface and a generally spherically shaped distal surface. The exemplary bone anchor assembly is a polyaxial bone screw designed for posterior implantation in the pedicle or lateral mass of a vertebra. In this regards, the proximal head 28 of the bone anchor 24 engages the distal end 38 of the receiver member 22 in a ball and socket like arrangement in which the proximal head 28, and thus the distal shaft 24, can pivot relative to the receiver member 22. The distal surface of the proximal head 28 of the bone anchor 24 and the mating surface within the distal end 38 of the receiver member 22 may have any shape that facilitates this ball and socket like arrangement, including, for example, spherical (as illustrated), toroidal, conical, frustoconical, and any combinations of these shapes.

The distal shaft 30 of the bone anchor 24 may be configured to engage bone and, in the illustrated embodiment, includes an external bone engaging thread 40. The thread form for the distal shaft 30, including the number of threads, the pitch, major and minor diameter, and thread shape, may be selected to facilitate connection with bone. Examples of exemplary thread forms are disclosed in U.S. patent application Ser. No. 13/110,378, filed May 18, 2011, which is incorporated herein by reference. Alternatively, the distal shaft 30 may include other structures for engaging bone, including a hook. The distal shaft 30 of the bone anchor 24 may be cannulated, having a central passage or cannula extending the length of the bone anchor to facilitate delivery of the bone anchor over a guide wire in, for example, minimally invasive procedures. The other components of the bone anchor assembly, including the closure member 26, the receiver member 22, and the compression member 50 (discussed below) may be cannulated or otherwise have an opening to permit the respective component to be delivered over a guide wire.

Continuing to refer to FIGS. 3-5, the proximal end 32 of the receiver member 22 of the exemplary primary bone anchor assembly 14 includes a pair of spaced apart arms 34A, 34B defining the U-shaped recess 36 therebetween for receiving a spinal fixation element. The distal end 38 of the receiver member 22 is generally cylindrical in shape and includes distal end surface 40 which is generally annular in shape defining a circular opening through which at least a portion of the bone anchor 24 extends. For example, the distal shaft 30 of the bone anchor 24 may extend through the opening. Each arm 34A, 34B of the proximal end 32 of the receiver member 22 extends from the distal end 38 of the receiver member 22 to a free end. The outer surface of each arm 34A, 34B may include a feature, such as a recess, dimple, notch, projection, or the like, to facilitate connection of the receiver member 22 and, thus, the primary bone anchor assembly 14, to instruments or other implants, such as connectors 18, 118. In the exemplary embodiment, for example, the outer surface of each arm 34A, 34B includes an arcuate groove 42A, 42B at the respective free end of the arms. Such grooves are described in more detail in U.S. Pat. No. 7,179,261, which is incorporated herein by reference.

The proximal end 32 of the receiving member 22 may be configured to receive a closure mechanism, such as an internal set screw (closure mechanism 26) or an external cap or nut. For example, the interior surface of each arm 34A, 34B may include a feature, such as a recess, dimple, notch, projection, thread or the like, to facilitate connection of the closure mechanism 26 to the receiver member 22. In the exemplary embodiment, for example, the interior surface of each arm 34A, 34B includes an internal thread 44 on the interior surface of each arm 34A, 34B for engaging the closure mechanism 26. In the exemplary embodiment, the thread starts at the free, proximal end and extends distally along at least a portion of the length of the arms 34A, 34B.

The closure mechanism 26 in the exemplary embodiment is an internal set screw having an external thread that engages the internal thread of the receiver member to capture a spinal fixation element within the recess 36 of the receiver member and, when fully tightened, to fix the spinal fixation element relative to the receiver member 22. Alternatively, the closure mechanism may be dual closure mechanism having an inner and an outer set screw, such as, for example, the Expedium Dual Innie Polyaxial Screw available from DePuy Spine, Inc. of Raynham, Mass. In addition, the closure mechanism may be a non-threaded twist-in cap, such as, for example, the Monarch Typhoon Cap available from DePuy Spine, Inc. of Raynham, Mass., and described in U.S. Pat. No. 6,755,829, incorporated herein by reference.

The exemplary primary bone anchor assembly 14 may be used with a spinal fixation element such as a rigid spinal rod 12. The spinal rod may be constructed from titanium, titanium alloys, stainless steel, cobalt chrome, PEEK, or other materials suitable for rigid fixation. Alternatively, the spinal fixation element may be a dynamic stabilization member that allows controlled mobility between the instrumented vertebrae.

The exemplary bone anchor assembly is a rigid polyaxial screw in which the primary bone anchor 14 is fixed, rather than mobile, when the spinal fixation element is fixed to the receiver member 22 of the primary bone anchor assembly 14. The spinal fixation element may either directly contact the proximal head 28 of the bone anchor 24 or may contact an intermediate element, e.g., a compression member 50, interposed between the spinal fixation element and the proximal head 28 of the bone anchor 24 to compress the distal outer surface of the proximal head 28 into direct, fixed engagement with the distal inner surface of the receiver member 22 when the spinal fixation element is fixed to the receiver member 22 of the primary bone anchor assembly 14 by the closure mechanism 26. In alternative embodiments, the primary bone anchor assembly may be a mobile screw in which the proximal head 28 of the bone anchor 24 can move relative to the receiver member 22 when the spinal fixation element is fixed to the receiver member 22. An exemplary mobile polyaxial screw is described is U.S. Patent Application Publication No. US 2011-0093021, which is hereby incorporated herein by reference. Alternatively, the bone anchor assembly may be a monoaxial screw, a favored angle screw or a uniplanar screw.

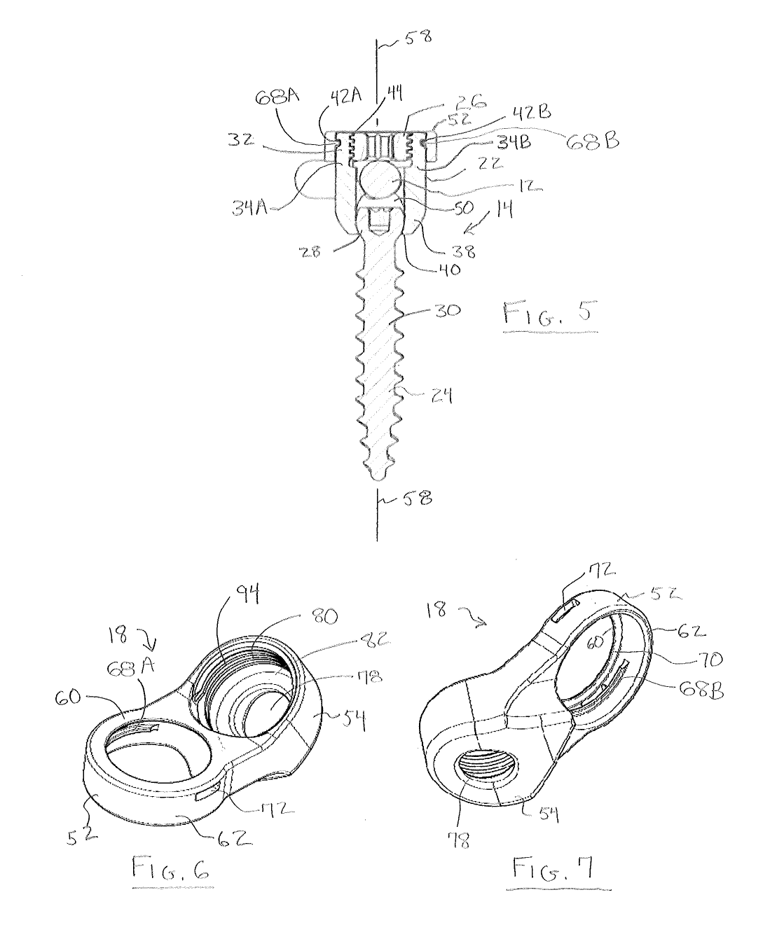

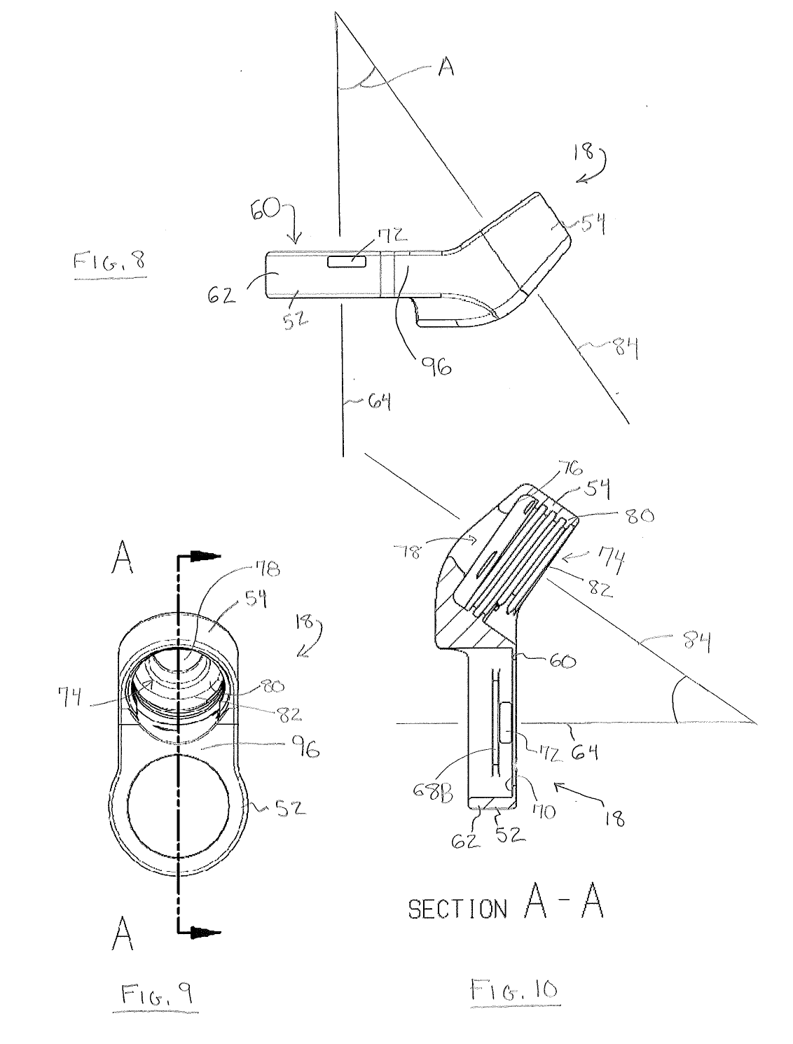

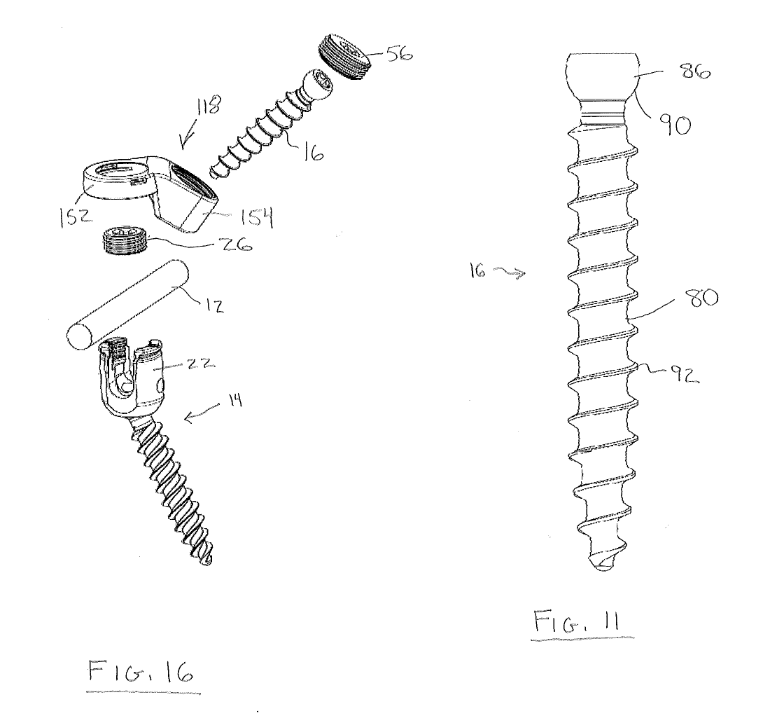

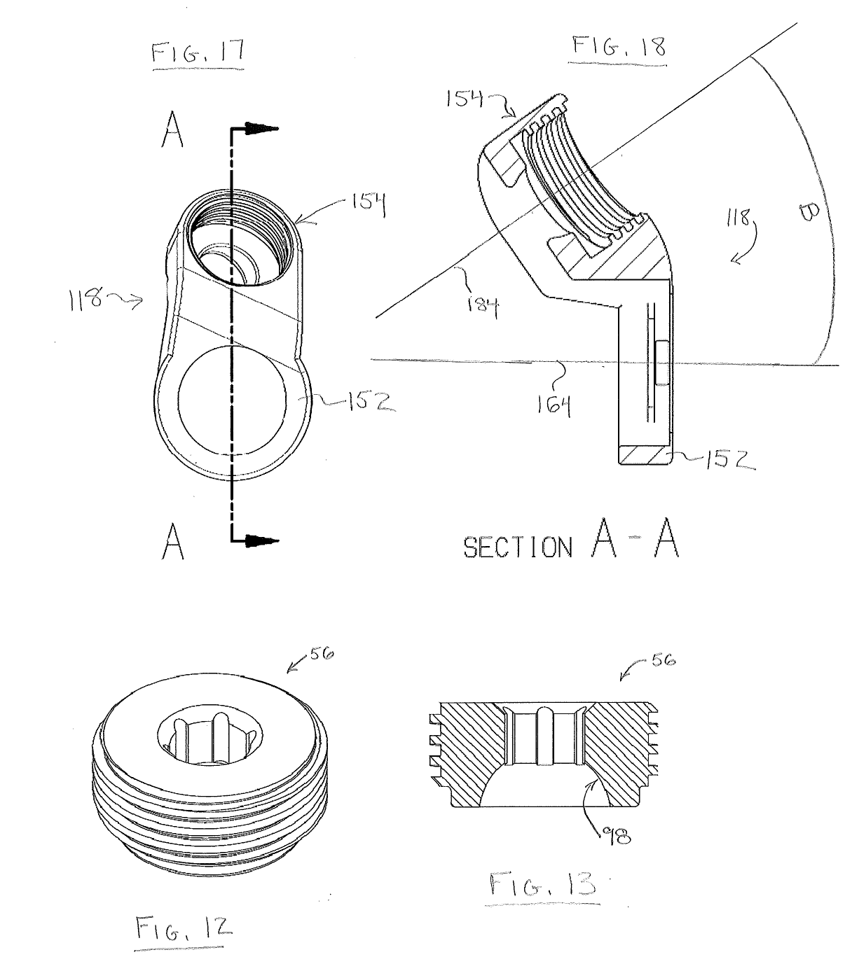

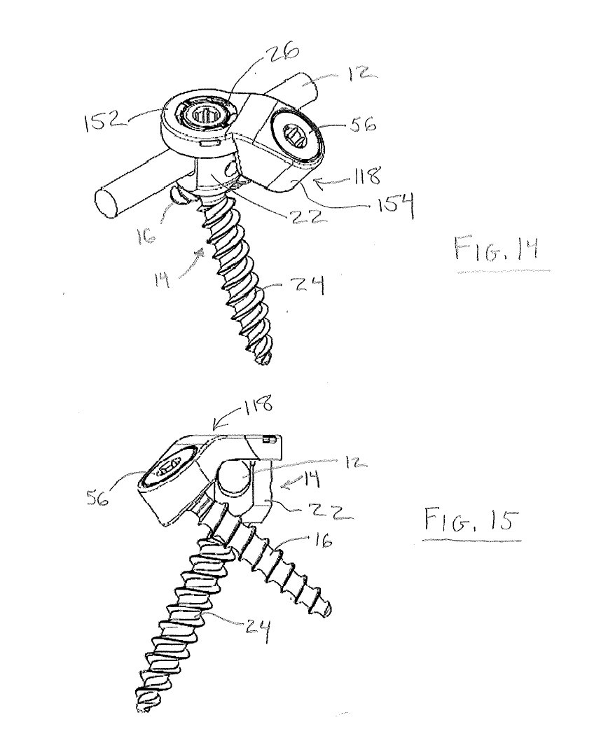

Continuing to refer to FIGS. 3-5 and also referring to FIGS. 6-10, an exemplary connector 18 includes a first component 52 engageable to the receiver member 22 of a primary bone anchor assembly 14, a second component 54 connected to the first component 52 for receiving the secondary bone anchor 16 in a plurality of angular orientations, and a second closure mechanism 56 engageable with the second component 54 to fix the secondary bone anchor 16 at a selected angular orientation relative to the second component 54.

In the exemplary embodiment, the first component 52 may be rotatably adjustable relative to the receiver member 22. In particular, the first component 52 is rotatably adjustable about the longitudinal axis 58 of the receiver member 22. The first component is generally ring shaped including a proximal ring 60 and an annular side wall 62 extending distally from the proximal ring 60. The annular side wall 62 may have an inner diameter sized to permit positioning of the first component 52 about the receiver member 22 of primary bone anchor assembly 22. The first component 52 includes a longitudinal axis 64 intersecting a center of the proximal ring 60. When the first component 52 is positioned about the receiver member 22 the longitudinal axis 64 if the first component 52 and the longitudinal axis 58 of the receiver member 22 are coincident.

The inner surface 66 of the annular side wall 62 of the first component 52 may include a pair of diametrically opposed, spaced apart projections 68A, 68B to facilitate connection and, subsequently, retention of the first component 52 to the receiver member 22 of the primary bone anchor assembly 14. Preferably, the projections 68A, 68B and the recesses 42A, 42B are complementary in shape to permit positioning of each projection 68A, 68B within a respective recess 42A, 42B and thereby inhibit axial motion of the first component 52 (e.g., motion parallel to the longitudinal axis 58 of the receiver member 22) relative to the receiver member 22 of the primary bone anchor assembly 14. In the exemplary embodiment, the projections 68A, 68B are arcuate in shape having a length sized to fit between the arms 34A, 34B of the receiver member 22. In this manner, the first component 52 may be oriented to position each projection 68A, 68B between the arms 34A, 34B of the receiver member 22, advanced distally, and rotated to position each projection 68A, 68B at least partially in a respective one of the recesses 34A, 34B. The rotational position of the first component 52 relative to the may be adjusted to orient the second component 54 at a desired position to accommodate the desired trajectory of the secondary bone anchor 16. In the exemplary embodiment, securing the closure mechanism 26 to the receiver member 22 fixes the rotational position of the first component 52 relative to the receiver member 22. In particular, rotation of the closure mechanism 26 into engagement with the internal thread 44 on the arms 42A, 42B may cause radially outward deflection or splay of the arms 42A, 42B. This outward deflection may create an interference between the outer surface of the receiver member 22 and the inner surface of the annular side wall 62 of the first component 52, thereby securing the rotational position of the first component 52 relative to the receiver member 22.

The proximal ring 60 preferably has a diameter that is less than the outer diameter of the receiver member 22 of the primary bone anchor assembly 14. In this manner, the distal surface 70 of the proximal ring 60 may engage the proximal surface of the receiver member 22 to inhibit axial motion of the first component 52 in the distal direction relative to the receiver member 22. The first component 52 may include one or more openings or the like to facilitate connection of an instrument to the first component 52. In the exemplary embodiment, the first component 52 includes a pair of diametrically opposed, spaced apart slots 72 in the annular side wall 62 for receiving projections from an instrument.

Continuing to refer to FIGS. 3-10, the second component 54 of the exemplary connector 18 forms a socket 74 to receive the secondary bone anchor 16 in a plurality of orientations. The socket 74 may be generally cylindrical in shape having a distal end wall 76 with a distal opening 78 therein and side walls 80 extending proximally from the distal end wall 76 at an orientation generally perpendicular to the distal end wall 76. The side walls 80 terminate at a proximal end of the socket 74 to define a proximal opening 82 in the socket 74. To facilitate top loading insertion of the secondary screw 16 into the socket 74 (e.g., insertion from the proximal to the distal direction) and subsequent retention of the proximal head 86 of the secondary screw 16, the distal opening 78 may have a diameter less than a diameter of the proximal opening 82. The socket 74 includes a longitudinal axis 84 oriented perpendicular to the distal end wall 76 and intersecting a center of the distal opening 78 and a center of the proximal opening 82.

The secondary bone 16 anchor includes a proximal head 86 and a bone engaging distal shaft 88 having one or more bone engaging threads. The proximal head 86 of the secondary bone anchor 16 in the exemplary embodiment is generally in the shape of a truncated sphere having a planar proximal surface and a generally spherically shaped distal surface 90. In the exemplary embodiment, the proximal head 86 of the secondary bone anchor 16 engages the socket 74 formed by the second component 54 of the exemplary connector 18 in a ball and socket like arrangement in which the proximal head 86, and thus the distal shaft 88, can pivot relative to the second component 54. The distal surface 90 of the proximal head 86 of the secondary bone anchor 16 and the mating surface of the within the socket 74 may have any shape that facilitates this ball and socket like arrangement, including, for example, spherical (as illustrated), toroidal, conical, frustoconical, and any combinations of these shapes. In this regard, the inner surface of the distal end wall 76 about the distal opening 78 may be generally spherical in shape. In the exemplary embodiment, the secondary bone anchor 16 can pivot through a cone of angulation centered about the longitudinal axis 84 of the socket 74.

The distal shaft 88 of the secondary bone anchor 16 may be configured to engage bone and, in the illustrated embodiment, includes an external bone engaging thread 92. The thread form for the distal shaft 88, including the number of threads, the pitch, major and minor diameter, and thread shape, may be selected to facilitate connection with bone. The thread form of the distal shaft 88 may be similar to the thread form of the distal shaft 30 of the bone anchor 2, discussed above.

The socket 74 formed by the second component 54 of the exemplary connector 18 may include an internal thread 94 proximal the proximal opening 82 for engagement with a complementary external thread provided on the second closure mechanism 56, which, in the exemplary embodiment, is an internal set screw. Referring to FIGS. 12 and 13, the closure mechanism 56 includes a hemi-spherically shaped recess 98 that opens at the distal surface of the second closure mechanism 56 to engage the proximal head 86 of the secondary bone anchor 16. The hemi-spherically shaped recess 98 may be complementary in shape to the hemi-spherically shape proximal head 86 of the secondary bone anchor 16. The closure mechanism 56, when fully tightened, engages the proximal head 86 of the secondary bone anchor 16 to force the distal surface 90 of the proximal head 86 into engagement with the distal end wall 76 of the socket 74 to lock the secondary bone anchor 16 at a selected angle relative to the second component 54.

Continuing to refer to FIGS. 3-10, and in particular FIG. 8, the first component 52 may be connected to the second component 54 at a bend zone 96 that permits selective adjustment of angle A between the longitudinal axis 84 of the socket 74 and the longitudinal axis 64 of the proximal ring 60. By adjusting the angle A between the longitudinal axis 84 and the longitudinal axis 64, the cone of angulation for the secondary bone anchor 16 can be adjusted to facilitate placement of the secondary bone anchor 16 at the desired trajectory in bone. Prior to bending, the angle A may be between 25° and 45°, and, in the illustrated embodiment is approximately 35°. By bending the second component 54 relative to the first component 52 at the bend zone 96, the angle A between longitudinal axis 84 and longitudinal axis 64 may be increased to 90° or any angle between the initial angle (e.g., 35°) and 90°.

In use, the first component 52 of the connector 18 can be connected to the primary bone anchor assembly 14 after placement of the first bone anchor assembly 14 into bone, e.g. the sacrum, and positioning of a spinal rod 12 within the primary bone anchor assembly 14. The first component 52 can be rotated relative to the primary anchor assembly 14 to select the desired position for the second component 54 based on the desired trajectory for the second bone anchor 16. Once the desired rotational position is selected, the secondary bone anchor 16 may be positioned through the socket 74 of the second component 54 into bone, e.g., the sacrum. The second closure mechanism 56 can be connected to the second component 54 to secure the secondary bone anchor 16 relative to the socket 74. The first closure mechanism 26 can be connected to the primary bone anchor assembly 14 to secure to first component 52 of the connector 18 and the spinal rod 12 to the first bone anchor assembly 14 and to fix the position of the bone anchor 24 relative to the receiver member 22 of the first bone anchor assembly 14.

FIGS. 14-17 illustrate another embodiment of a connector 118 for connecting a secondary bone anchor 16 to one of the primary bone anchor assemblies 14 of the spinal fixation system 10. The connector 118 is generally analogous in construction to the connector 18 described above and includes a first component 152 engageable to the receiver member 22 of a primary bone anchor assembly 14, a second component 154 connected to the first component 152 for receiving the secondary bone anchor 16 in a plurality of angular orientations, and a second closure mechanism 56 engageable with the second component 154 to fix the secondary bone anchor 16 at a selected angular orientation relative to the second component 154. The longitudinal axis 164 of the first component 152 of the connector 118 intersects the longitudinal axis 184 of the second component 154 at a point proximal to the connector 118 and the first bone anchor assembly 14. In contrast, the longitudinal axis 64 of the first component 52 of the connector 18 intersects the longitudinal axis 84 of the second component 54 at a point distal to the connector 18 and the first bone anchor assembly 14. In this manner, the connector 18 and connector 118 allow the surgeon more selections between trajectories for the secondary bone anchor 16. The angle B between the longitudinal axis 164 of the first component 152 of the connector 118 and the longitudinal axis 184 of the second component 154 may be similar to the angle A described in connection with exemplary connector 18.

While the devices and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

What is claimed:

1. A connector for connecting a second bone anchor to a first bone anchor, the connector comprising:
a first component engageable to the first bone anchor and rotatably adjustable relative to the first bone anchor,
a second component connected to the first component and forming a socket to receive the second bone anchor in a plurality of angular orientations, and
a closure mechanism engageable with the second component to fix the second bone anchor at a selected angular orientation relative to the second component.

2. The connector of claim 1, wherein the first component is generally ring shaped.

3. The connector of claim 2, wherein the first component comprises a proximal ring and an annular side wall extending distally from the proximal ring, the annular side wall having an inner diameter sized to permit positioning of the ring about a portion of the first bone anchor, the proximal ring including a longitudinal axis intersecting a center of the proximal ring.

4. The connector of claim 3, wherein an inner surface of the annular side wall of the first component includes a pair of diametrically opposed, spaced apart projections for positioning within complementary shaped recesses in the first bone anchor.

5. The connector of claim 4, wherein the socket is generally cylindrical in shape having a distal end wall with a distal opening therein and side walls extending proximally from the distal end wall at an orientation perpendicular to the distal end wall, the side walls terminating at a proximal end of the socket to define a proximal opening in the socket, the distal opening having a diameter less than a diameter of the proximal opening, the socket including a longitudinal axis oriented perpendicular to the distal end wall and intersecting a center of the distal opening.

6. The connector of claim 5, wherein inner surface of the distal end wall about the distal opening is generally spherical in shape.

7. The connector of claim 5, wherein the socket includes an internal thread proximal the proximal opening and wherein the closure mechanism includes an external thread for engaging the internal thread of the socket.

8. The connector of claim 5, wherein the first component is connected to the second component at a bend zone that permits selective adjustment of an angle between the longitudinal axis of the socket and the longitudinal axis of the proximal ring.

9. A spinal fixation system comprising:
a plurality of spinal rods,
a plurality of primary bone anchor assemblies for connecting one of the spinal rods to bone, the primary bone anchor assemblies each including a proximal receiver member for receiving a spinal rod, a distal bone anchor for engaging bone, and a closure mechanism for fixing the spinal rod relative to the receiver member,
a secondary bone anchor, and
a connector for connecting the secondary bone anchor to one of the primary bone anchor assemblies, the connector including
a first component engageable to the receiver member of one of the first bone anchor assemblies, the first component being rotatably adjustable relative to the receiver member wherein securing the closure mechanism to the receiver member fixes the rotational position of the first component relative to the receiver member,
a second component connected to the first component and forming a socket to receive the secondary bone anchor in a plurality of angular orientations, and
a second closure mechanism engageable with the second component to fix the secondary bone anchor at a selected angular orientation relative to the second component.

10. The system of claim 9, wherein the first component is generally ring shaped.

11. The system of claim 10, wherein the first component comprises a proximal ring and an annular side wall extending distally from the proximal ring, the annular side wall having an inner diameter sized to permit positioning of the ring about the receiver of one of the first bone anchor assemblies, the proximal ring including a longitudinal axis intersecting a center of the proximal ring.

12. The system of claim 11, wherein an inner surface of the annular side wall of the first component includes a pair of diametrically opposed, spaced apart projections and wherein the receiver members of the first bone anchor assemblies each include a pair of diametrically opposed, space apart recesses, wherein the projections and recesses are complementary in shape to permit positioning of each projection within a recess and thereby inhibit axial motion of the first component relative to the receiver member.

13. The system of claim 12, wherein the socket is generally cylindrical in shape having a distal end wall with a distal opening therein and side walls extending proximally from the distal end wall at an orientation perpendicular to the distal end wall, the side walls terminating at a proximal end of the socket to define a proximal opening in the socket, the distal opening having a diameter less than a diameter of the proximal opening, the socket including a longitudinal axis oriented perpendicular to the distal end wall and intersecting a center of the distal opening and a center of the proximal opening.

14. The system of claim 13, wherein inner surface of the distal end wall about the distal opening is generally spherical in shape and wherein the secondary bone anchor includes a proximal head and a bone engaging distal shaft, the proximal head having a spherically-shaped distal surface.

15. The system of claim 14, wherein the socket includes an internal thread proximal the proximal opening and wherein the second closure mechanism includes an external thread for engaging the internal thread of the socket.

16. The system of claim 13, wherein the first component is connected to the second component at a bend zone that permits selective adjustment of an angle between the longitudinal axis of the socket and the longitudinal axis of the proximal ring.

17. A spinal fixation system comprising:
a plurality of spinal rods,
a plurality of primary bone anchor assemblies for connecting one of the spinal rods to bone, the primary bone anchor assemblies each including a bone anchor, a proximal receiver member for receiving a spinal rod to be coupled to the bone anchor, and a closure mechanism, the bone anchor having a proximal head and a distal shaft configured to engage bone, the receiver member having a proximal end having a pair of spaced apart arms defining a recess therebetween, a distal end having a distal end surface defining opening through which at least a portion of the bone anchor extends, and the closure mechanism positionable between and engaging the arms to capture a spinal rod within the receiver member and fix the spinal rod with respect to the receiver member,
a secondary bone anchor having a proximal head and a bone engaging distal shaft, and
a connector for connecting the secondary bone anchor to one of the primary bone anchor assemblies, the connector including
a ring having an annular side wall having an inner diameter sized to permit positioning of the side wall about a portion of a receiver member of one of the bone anchor assemblies, the ring being rotatably adjustable relative to the receiver member, wherein securing a closure mechanism to the receiver member fixes the rotational position of the ring relative to the receiver member,
a socket connected to the ring, the socket having an internal seat configured to engage the proximal head of the secondary bone anchor and to permit pivoting of the distal shaft of the secondary bone anchor in a plurality of angular orientations, and
a second closure mechanism engageable with the socket to fix the distal shaft of the secondary bone anchor at a selected angular orientation relative to the socket.

18. The system of claim 17, wherein the bone anchors of the primary bone anchor assemblies are pivotable relative to a respective receiver member prior to fixation of the spinal rod to the receiving member.

19. The system of claim 17, wherein the bone anchors of the primary bone anchor assemblies are fixed relative to a respective receiver member.

20. The system of claim 17, wherein the outer surface of each arm of the receiver members of the primary bone anchor assemblies includes a recess and wherein an inner surface of the annular side wall of the ring includes a pair of diametrically opposed, spaced apart projections, the projections and recesses being complementary in shape to permit positioning of each projection within a recess and thereby inhibit axial motion of the ring relative to the receiver member.
